(19) United States
(12) Patent Application Publication
(10) Pub. No.: US 2010/0322874 A1
(43) Pub. Date: Dec. 23, 2010

(54) ABSORBANT/COMPLIANT SOLUTION-FREE CLEANING AND MULTIACTIVE COMPOSITIONS

(76) Inventor: Hans O. Ribi, Hillsborough, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/446,552

(22) PCT Filed: Oct. 23, 2007
(86) PCT No.: PCT/US2007/022499

§ 371 Date: Aug. 30, 2010

Related U.S. Application Data
(60) Provisional application No. 60/853,832, filed on Oct. 23, 2006.

(51) Int. Cl. A61K 8/72; A61K 8/73; A61K 8/92; A61K 8/81; A61K 8/89; C11D 17/00; A61Q 17/04; A61K 38/12; A61P 31/04

(52) U.S. Cl. 424/59; 510/138; 510/100; 510/403; 510/396; 514/2.9; 510/384; 510/383

(57) ABSTRACT

Compliant liquid-free cleaning compositions and methods for using the same are provided. A composition that is pliable and putty-like that can be rolled and pressed onto dirt to clean the dirt off of a surface is provided. The composition can be used on hands, household surfaces, countertops, or any item displaying dirt. The invention eliminates the need for using water-based or other liquid cleansers.

### CROSS-REFERENCE TO RELATED APPLICATIONS

Pursuant to 35 U.S.C. §119(e), this application claims priority to the filing date of: U.S. Provisional Patent Application Ser. No. 60/853,832 filed Oct. 23, 2006, the disclosure of which application is herein incorporated by reference.

### INTRODUCTION

Many forms of infectious disease, such as the common cold, are contracted and spread to others, primarily by the hands. Conventional medical wisdom teaches that the best way to prevent contraction and transmission of most common bacterial and viral infections is by thoroughly and often cleansing the hands throughout the day, and primarily before touching one's face or preparing food.

Cleansing and sanitizing an individual's hands using warm soapy water is generally effective for removing “germs” that have accumulated on hands throughout the day. For additional protection, an antibacterial hand soap may be utilized in lieu of conventional soap. Washing and rinsing of the hands is sometimes followed by applying a topical sterilant such as for example, a denatured alcohol, an anti-bacterial hand gel or anti-bacterial hand lotion to further protect the hands. Although these measures will help to control the spread of many pathogens residing on the skin, they are ineffective if not performed routinely and methodically. Unfortunately, routine use of antibacterial soaps, lotions and gels may cause chapping of the skin or other undesirable reactions, especially for those persons being sensitive to the active ingredients found in these products.

As such, there is continued interest in the development of new sanitizing compositions and methods.

### SUMMARY

A composition that is pliable and putty-like that can be rolled and pressed onto dirt to clean the dirt off of a surface is provided. The composition can be used on hands, household surfaces, countertops, or any item displaying dirt. The invention eliminates the need for using water-based or other liquid cleansers. The composition extracts dirt from a surface and encapsulates the dirt into its own matrix. The residual dirt is then contained within the compound and is not released. The composition can contain agents that soften hands such as lotions, fragrances that offer a refreshed smell and feeling, and antibacterial agents to ensure that a surface is both clean and antiseptic. The inventive composition can contain a color-change agent to promote proper use by indicating a color change when the composition is either activated or at an optimal texture for removing dirt from a surface. Other color additives or agent can comprise the composition to indicate when a cleaning composition amount has been fully utilized and is ready to discard.

Embodiments of the composition require no water, absorb a high percentage by weight of dirt, and can be formulated for general use or specific usages, have plural elements for formulation and usage, are economic to manufacture and use, are made with non-toxic base materials. Embodiments of the compositions can be food grade for certified for contact with food, can pick up a variety of dirt, greases, grimes, bacteria etc., can simultaneously remove dirt and leave behind desired compositions such as hand softeners, anti-bacterial agents, fragrances, does not remove permanent colors, can remove and absorb odors and/or stains, can be softened to activate or pre-softened and already activated; absorbs and encapsulates contamination; or the like.

The invention is comprised with a composition with single and/or multiple elements that provide the composition with one or more properties that enable a simple waterless or solvent-less means for cleaning a surface. The element properties described are intended to illustrate exemplary features of the composition and are not limited to only those described.

The invention comprises a composition with the element properties of being absorbent/adherent to a contaminating species adherent to a surface. The absorbent/adherent properties provide the composition to directly adhere to and stick contaminating species to its surface. The composition further possesses the element property of being moldable/compliant. The moldable/compliant properties provide the composition with the ability to contact and conform to a surface providing an intimate interface with even deep cavity-like surface features that often contain trapped contaminating species. The composition further possesses the element properties of entrapping/encapsulation. The entrapping/encapsulating properties provide a means to bind a contaminating species into the bulk phase of the composition such that the contaminating species remains adhered and contained within the composition and cannot be further removed from the composition during normal use. The composition can further possess the element property of a high loading capacity of a contaminating species. The high loading capacity property provides the composition with the ability to collect and carry a significant percentage by weight or molar equivalent of its mass such that a particular allotment of the composition can be used and reused numerous times for cleaning prior to it becoming saturated and no longer effective.

Compared and contrasted with hand sanitizers, cleaning compositions disclosed simultaneously sanitize and remove dirt and/or grime from a surface. Hand sanitizers are usually in a liquid form and can cause skin irritation due to the solvents they possess. Although the solvents utilized in a hand sanitizer may kill a percentage of micro-organisms on a surface, they do not remove any residual killed, attenuated, or remaining living organisms. By contrast, the composition disclosed, can both kill micro-organisms as well a remove any killed, attenuated, or remaining living organisms. Furthermore, entrapment of remaining living micro-organisms into the matrix of the composition will lead to rapid death of the micro-organism due to the presence of concentrated anti-bacterial and/or anti-viral active ingredients with in the composition formulation. Hand sanitizers effectively distribute and smear in dirt and grime on fingers and surfaces whereas the composition disclosed effectively remove dirt from hands and a surface. Collectively, the properties of the composition provide for a means of adherently removing dirt, grime and micro-organisms as well as disinfecting the surface and killing micro-organisms within the matrix of the composition compared to a hand sanitizer that can only effectively kill a portion of micro-organisms on the surface.

Whereas a hand sanitizing solution can cause skin to dry and crack, the composition can replace natural oils and rejuvenate dry, parched and cracked skin rather than contribute to the problems caused by over-the-counter and prescription hand sanitizers. The addition of therapeutic oils and balms delivered to skin during the cleaning process can significantly assist in the healing damaged skin.

Compared and contrasted with liquid cleaners, a cleaning solution requires the assistance of a wipe cloth or sponge for active removal from a surface. Wipe cloths require subsequent cleaning or disposal-effectively after each cleaning cycle. Cleaning wipes and sponges can be abrasive even in the hydrated state compared with formulations of the disclosed composition that can be prepare entirely free of abrasives. The cleaning mechanism for a wipe involves a “swiping action” for dirt removal compared with the composition that involve an adhesive “lift off” action. Wipes/liquid cleaners require significant percentages by weight of water for effective cleaning action. Typical cleaning solutions are often in excess of 90% water. Typical wipe formulations include 3% by weight solution of an alcohol. By contrast, the composition of the invention can be effectively dry of water or solvents and yet retain complete cleaning activity. Wipes retain dirt and grim on their surface with little or no internalization of the contamination into the wipes matrix. Sponges can absorb a quantities of dirt and grim, but need to be constantly cleaned and rinsed to remain effective. By contrast, the composition can internalize a significant percentage by weight from its surface into its matrix thereby providing a significantly prolonged cleaning ability compared with a wipe.

Adhesive and rubberized rollers that are commercially available require on-going removal of dirt, grime and contamination from their surface for effective cleaning. Typical rubberized rollers require subsequent cleaning with running water to effectively remove adhered dirt or grim. Adhesive tape rollers require removal and disposal or a tape layer for continued use. Likewise, roller type cleaning devices become highly ineffective when contacted by grease and oils. In comparison, compositions of the invention, e.g., as disclosed herein, can be used continually while dirt, grim, grease and oils become internalized. On-going subsequent surface cleaning and inactivity does not become an issue when utilizing the composition.

Compositions of the invention can be formulated for immediate disposability or prolonged use and reusability. Disposable compositions can be formulated using lower cost bulk components whereas semi-disposable or prolonged use formulations can take advantage of higher-quality longer-lasting components.

The molding and modeling characteristics of the composition provide it with an intrinsic entertainment/play value for children to encourage them to clean their fingers and skin. Unlike existing soaps, cleaners, wipes and sanitizers children can be entertained by simultaneously molding shapes and designs in the composition while the composition is acting to cleans and deposit a protective coating on fingers and skin. In addition, adding color change properties to the composition provide the composition with additional intrinsic interest to children as well as adults.

The molding properties and qualities of the composition further provide it with a means to be shaped into configurations that are advantageous to cleaning hard-to-get at cracks, grooves and corners and otherwise difficult to clean areas. Pre-molding the composition into a particular shape allows the composition to be inserted into deep and difficult spaces. Likewise, the composition can be immediately reshaped for other difficult to clean areas.

Compositions can be either softened by contact with fingers, modeling, pressing, rolling or any other kneading activity that may be utilized to soften and increase the adhesive properties of the composition. Alternatively, pre-softened compositions can be utilized that possesses moderately adhesive characteristics in a “pre-softened” state. The latter has the advantage of instantaneous utility while compositions requiring kneading have the advantage of exhibiting less adhesive characteristics until utilized and soften for activation. Various mineral oils, vegetable oils, soy-based oils, silicon oils and the like when added at concentrations above 5% increase the pliability and compliance of the composition. Typically softening oils will be added from 0.1% to 50%. More usually softening oils will be added between 1% and 40%. Most often, softening oils will be added between 5 and 20%.

### Industries and Sector Applications:

Industry, sector, and category applications can include, but are not limited to household; machine shop (e.g. grease and metal fragments); service sectors; schools, day care, and academic institutes; automotive detailing; hotels and spas; athletic workout facilities and sports clubs; medical institutional applications; pharmaceutical and wound care; industrial cleanup; space flight where aqueous cleaning is unacceptable; supermarket checkout and shopping carts; restaurant and fast food counters and cash registers; public restrooms; computer terminals and key boards that in particular should be avoided by water; public sanitation services; toy stores and various toy products; dry cleaning facilities for stain removal; surfaces that would be damage by water (e.g. photos and papers); art work and museums; zoos and family recreational; outdoor recreational and camping; military training, camp and battle environments; work shops where sawdust, metal particles, metal filings, and the like get deposited; hair and beauty salons where hair can be readily collected by the composition; travel sectors; cruise ships; airliners; office environments; manufacturing environments; schools and day care facilities where desks, chalkboards, dry boards and the like need cleaning; pet care; rest homes; laboratory cleanup and safety environments; food service and sales; automotive; movie theaters; bars; gas and service stations and rest stops; recreational theme parks; carpet and upholstery; fabrics and clothing; retail clothing outlets and returns; dry cleaning and cleaning services; skin care; health and beauty aids; household and window cleaning services; bio-hazardous cleanup and encapsulation: furniture care including dusting; paper and office supplies including erasers; and the like.

The liquid-free compliant/absorbant compositions find particular use in removing micro-organisms, germs, bacterial contamination, other pathogens such as viruses, and the like from fingers, skin, and various hard and soft surfaces. The composition can remove micro-organisms alone and/or work in combination with included sanitizing/germicide agents. The composition can be effective at either or micro-organism removal alone or in combination killing micro-organisms on contact by simultaneously. Additional germicides include, but are not limited to iodine, alcohols, hydrogen peroxide, peracetic acid, phenol, quaternary ammonium compounds. The compositions containing added alcohol as a sanitizer has a distinct advantage over liquid/gel type hand sanitizers since the ethanol in the putty/cleanser matrix does not cause drying of skin and is not available for consumption by a user such as a child that might inadvertently consume an alcohol-based hand sanitizer.

Sanitizing or germicidal agents are effective when added between 0.01% and 50%. More often concentrations between 0.05% and 40% will be of use. Usually, concentrations between 0.1% and 25% will find used. Most often concentrations between 0.5% and 10% are utilized.

### Surface Types and Geometries:

Surface types with contaminating elements included but are not limited to: smooth planar surfaces, textured surfaces, undulating surfaces, round surfaces, corners, grooves, patterned surfaces, cracked regions, grouted regions, shaped surfaces such as handles, puckered surfaces, surfaces with interstitial regions, tortuous surfaces, sanded or polished surfaces, compliant or hard surfaces, fiber or filament type surfaces, surfaces with undercuts or hard to get at spaces, or the like. Because the compositions disclosed are formulated to compliant selectively shaped, the composition can be used to access and clean an unusually wide range of surface textures and geometries compared with conventional cleaning substrates such as wipes, towels, and sponges.

### Composition Shapes and Configurations:

Product configurations can include a wide range of different forms that represent convenient to use sizes and shapes. Product shapes can include those that are easy to package, those that are easy and attractive to display in a package, sizes that are pre-determined for proper use and dose, those that are easy for a consumer to remove a recommended amount for use and the like. Shapes can include: bars, coated objects, rods, discs, coins, balls, rings, molded shapes for children, geometric shapes, drops, cylinder shapes, soap bar shapes to improve familiarity, licensed character shapes for children's applications, and the like.

Of particular interest are shapes that the composition can be packaged into. The composition can be pre-molded to and attractive, interesting or instructive shape that provides for an appealing and representative shape as seen by consumers prior to purchasing the product. New products can benefit greatly from packaging formats that assist in communicating to consumers what new features and properties that the product possesses. The composition can be formed during production into a representative shape that fits into a pre-formed transparent blister package or can be pres-molded directly into the blister packaging material. In either case, the molding characteristics of composition provide for unique packaging concepts and formats. In addition, since the composition can be used immediately as packaged, features on the package or limited areas of the composition itself can be expose for consumers to try the product directly on the store shelf.

### Dirt/Contamination Types:

Dirt and contamination types include, but are not limited to animal, mineral, and vegetable grease type; finger grease; grease spots; water spots; soap scum; insects; sand and fine grain dirt; carbon and graphite; pencil marks and certain ink types; grass stain; dry powders such as sand and chalk; water marks on dishes, mirrors, and classes; fragile items and surfaces such as artwork can be cleaned without using water or liquid cleansers;

### Household Cleaning Applications:

Bathroom cleaning applications include soap scum, mirror water marks, shower stalls, bath tubs and drains, toilets and toilet seats, counter tops, floors, faucets, cabinetry, appliances, walls, windows, spot removal on towels, hair and whiskers on sinks and counter tops, dirt and build up on grout, and any of a variety of dirt, grim and build-up encountered in a bathroom environment and the like.

Kitchen applications can include sinks and drains, faucets, counter tops, drain boards, windows, stove tops, oven and dishwasher doors, linoleum and wood floors, kitchen tile of all types, painted surfaces such as cabinetry, appliances, refrigerator insides and doors, pots and pans, dinner tables and chairs, dishes, cookware, utensils, cups and wine glasses, grease on cook tops or on or near grilling surfaces, and the like.

A wide range of different food, foot-traffic, cooking, finger, grease, waters spots, bacterial contaminations, and the like can be cleaned in a kitchen setting using the composition. It is of particular importance to reduce the incidents of food-borne contaminations in a kitchen setting where compositions possessing anti-bacterial components will find significant use.

Floor and furniture care applications include: picking up and removal of common dirt, grease spots, dust, sand, stains, oils, floor scuff marks from shoe heals and soles, oil stains, carpet spots and marks, floor mats, hard to get at corners and floor boards, shower floors, grout between tiles, cracks in hardwood floors, linoleum flooring, artificial grass, grooves in furniture including difficult to get at regions places and the like. By way of example and of interest are compositions that simultaneously clean and leave behind a thin furniture polish layer that results in both a clean and shinny surface on wood finishes and the like. Pet stains, hair, and odors can be effectively removed using the composition.

Clothing, business suits, blue jeans and denim products, sweaters, plush materials, silk and polyester fabrics, plush toys, pants, shirts, under garments, drapes, and other house-hold fabrics can be readily cleaned using the composition. Various typical stains include: grease spot removal, food stains, grass stains, pet stains, lip-stick stains or marks, perspiration stains, food stains and the like.

### Personnel and Medical Care Applications:

Personnel care applications include: hand soap, dandruff pickup, facial cleansers and grease removal, dirt and grease spot removal from arms, hands and legs, hair removal (depilatory applications), as a simple alternative to facial masks for deep facial cleansing, make-up removal coupled with skin cleansing and therapeutic skin coating, clean up after public outings, cleanup before mealtime, and the like. Compositions can include antibacterial agents for wound healing. The composition can be formulated to remove plaque from teeth or provide a means to deliver an oxidative tooth whitening agents. Since the composition can be formulated to be highly compliant such that the composition can be molded and nested on to teeth. Unlike tray type and strip type teeth cleaning appliances, the composition can be intimately formed around teeth making it highly effective as a whitening agent delivery system.

The composition can be formulated to remove allergens and other natural and unnatural substances that can induce an allergic or hypo-allergenic response. Allergens often collect on surface that may be attractive or adherent for certain common and non-common allergen types. The composition can assist in allergen removal from hard surfaces as well as compliant and skin surfaces thereby reducing the risk of allergen transfer to a susceptible individual. Pet dander, dust mites, pollen, as well as a wide range of other allergens can be effectively removed from surface utilizing the composition.

Composition applications can include facial grease removal and skin borne contaminations that may cause skin irritation, rashes, or other dermatological, problems. The composition can be formulated to remove type of facial grease as well as adhering grease and skin-pore buildups. By way of example, since the composition is both compliant and adherent, it can be useful for cleansing skin pores of black heads and other dermal irritations. The composition can further be used as a dry body wash whereby removing dirt, excess skin oil, body odor, dead skin, dandruff, and the like. In particular the composition may find use as a travel aid where it is difficult to shower, wash hands, or utilize any common means of bodily cleansing.

Medicinal delivery depot application can include using the composition to cleans a topical wound as well as deliver a metered amount of an antiseptic or anti-bacterial component. The composition can be loaded with a specified concentration of a topical medicinal agent such that contact with a wounded area can release the agent in a thin, but protective layer.

First aid antibiotics can be added to the composition to help promote wound healing when using the composition in contact with skin to cleans a region of skin that has a cut or abrasion. First aid antibiotics include but are not limited to Polymixin B sulfate, Bacitracin zinc, Neomycin sulfate, and the like. Antibiotics can be added to the composition alone or in combination to maximize the antibacterial effect.

The composition can be utilized to deliver a metered amount of an insecticide such as a mosquito repellent. Insect repellents in particular should be applied sparingly. The composition can find use for a variety of outdoor activities where both cleansing and application of an insect repellent is necessary. Many insect repellents are known to be harmful when used excessively especially when children are involved. The composition can be utilized as a metered delivery means to release only a thin film on to the surface of skin or clothing to provide necessary lasting protection on a surface needed without release of chemicals into the environment unnecessarily. Examples of mosquito repellents that can be added to the composition include, but are not limited to: ocimum selloi oil, novel compositions like those described in U.S. Pat. No. 6,355,264, and chrysanthemate, N,N-diethyl-m-toluamide (deet, commonly used), those described in U.S. Pat. No. 7,037,515 including ethyl 3-(N-butylacetylamino) propionate, p-menthane-3,8-diol, hydroxyethyl isobutyl piperidine carboxylate (1-piperidine carboxylic acid), and the like.

Additional beneficial additives that can aid in skin and topical skin care can include various aqueous and fat-based vitamins including vitamins A, B, B complexes, C, D, E, and A; anti-oxidants, minerals such as zinc; as well as other potential therapeutic components including herbal sources and the like. Likewise various cortisone or cortisone-like compounds can be utilize in the composition matrix to provide topical skin therapy for rashes and the like. Alternatively, anti-itch creams or ointments can be added to the composition to facilitate itch reduction and other irritations from insect bites or the like.

Local anesthetics and topical analgesics can be co-formulated in the composition such that use of the composition can provide pain therapeutic value during and after use. Examples include, but are not limited to: lanicaine, lidocaine, salicylates, other forms of aspirin, capsaicin as well as other forms of topical pain treatments.

### Remote Indoor and Outdoor Applications:

Fire damage, smoke spots, cigarette smoke stains on walls, furniture and ceilings, can be cleaned and cleansed using the composition. Remote areas or items where it is difficult to carry or afford a water-based or solvent-based cleaner can find utility using the composition. For example, stadium seats, lecture halls, movie theaters and the like can benefit utilizing the composition. Campers and out-door's people who wish to clean their hands and/or apply an insect repellent or hand softener can benefit utilizing the composition in particular where clean water may be scarce.

### Automotive Cleaning Applications:

Car care and automotive cleaning applications include fabrics, vinyl and leather coatings on seats, and gasoline or grease spills or spots on skin or clothing can be treated with the composition. Dust removal from car dashboards, finger grease removal from door handles, car doors and other parts of the car are readily accomplished using the composition. Wheels, tires, and hubcaps can be effectively cleaned by the composition. In particular, break dust and various oil and dirt deposits can be removed without the use of water or other solvents. Hydrophobic forms of the composition are preferred for removal of petroleum products.

### Fragrance and Aroma Delivery:

The composition can be formulated with a fragrance or aroma compounds, scents, menthols, perfumes or a wide range of aromatic substances that can be delivered by the composition during use. Of particular interest are formulations of compositions that actively release an aromatic substance upon the action of kneading, warming, or manipulation during use. The activation process can be utilized as a means to release a metered/desired amount of an entrapped aromatic substance either into the air or onto a surface. Release of the aromatic substance can serve as indicator of optimal activity for the composition during use or a depository property of the composition to add to the desired use of the product.

Aromatic additive can include, but are not limited to: sage, clove, eucalyptus, French lavender, juniper berry, lemon, coriander, cedar wood and green coconut—pure organic scent, neroli, flower fragrances, root fragrances, jasmine and mandarin, cypress, pure and organic, orange, patchouli, peppermint, spearmint, pink grapefruit, rosemary, Siberian fir, tea tree or the like.

Example Surfaces that can be Cleaned:

Any of a wide range of hard, pliable, elastic, or other materials can be effectively cleaned using the composition. Materials that can be cleaned include, but are not limited to: plastics, wood, fabrics, skin, metals, ceramic and porcelain, polymer surfaces, paint coatings, powder coated metals, paper, printed paper, rubber, concreted and plaster, stone, surfaces printed with ink, plastic laminate coatings, glass, protein surface such as finger nails and hair, vinyl surfaces, plaster, and a wide range of other common or technical materials.

Plastic surfaces that can be cleaned using the invention can include, but are not limited to: polyvinyl chloride (PVC), various polyolefins such as polypropylene and polyethylene, high density polyethylene (HDPE), low density polyethylene (LDPE), cross-linked high-density polyethylene (XLPE), softened acrylic, ABS, thick Kapton™ tape materials, Teflon® (polytetrafluoroethylene (PTFE), tetrafluoroethylene TFE and fluorinated ethylene polyproplyene FEP)-based materials, brand names such as Kydex, polystyrene, thermoplastic polyesters, nylon, styrene-butadiene, epoxy casts, polybutylene, TPX (poly(methyl pentene), terephtalate polyethylene (PET), PETE, PETF, polyethylene teraphthalate G copolymer (PETG), polysulfone (PSF), polyutethane (PUR) Thermanox™ (TMX), polymethylmethacrylate, and the like. Strong flexible plastics such as polycarbonate are often desirable. Polycarbonate can be thermoformed, pressure formed, and injection molded.

Other exemplary plastics may include, but are not limited to: ethylenechlorotrifluoreethylene (ECTFE), ethylentetrafluorethylene (ETFE), polinvinylidene fluoride (PVDF), ethylene-propylene rubber (EPR), silicone rubber (SI), Alcryn® thermoplastic rubber (TPR), HT thermoplastic rubber (HTPR), Santoprene® thermoplastic rubber (TPR), LSOH crosslinked compounds, LSOH thermoplastic compounds, methylvinyletherfluoralkoxy (MFA), perflouroalkoxy (PFA), thermoplastic polyester elastomer (TPE), polyimide (Kapton®), polyurethane (PUR), polyvinyl chloride 105° C. (PVC), polyvinyl chloride 70° C. (PVC), low temperature polyvinyl chloride (LTPVC), oil resistant Polyvinyl chloride (OR PVC), semirigid polyvinyl (SR PVC), polyvinyl chloride polyurethane (PVC PUR), and the like.

### DETAILED DESCRIPTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Composition Type A—Polymer Blended Based:

The basic composition contains poly-isobutylene as a thick, viscous, and adhesive polymer composite; calcium carbonate as an inert filler that helps to thicken, de-tackify, increase volume and reduce cost; and an emulsifier such as lecithin to facilitate dispersion, maintain a single phase and provide and overall consistency. The basic formulation can be modified by admixing one or a variety of additional compounds to further endow the composition with a particular property and/or quality for cleaning a contaminating substance from a particular surface.

In certain embodiments, the basic composition will contain about 1 to about 95% iso-butylene, such as from about 5 to about 75%, e.g., from about 10 to about 60% and including from about 25 to about 50% iso-butylene in the matrix. The final concentration will depend on the application of interest and balancing of other composition components.

In certain embodiments, the basic composition will contain from about 1 to about 95% calcium carbonate or a like-kind inert filler. In certain embodiments, the basic composition contains from about 5 to to about 75% filler in the matrix, such as from about 10 to about 60%, and including from about 25 to about 50% filler in the matrix. The final concentration will depend on the application of interest and balancing of other composition components.

In certain embodiments, the basic composition contain from about 0.1 to about 50% lecithin or a like-kind emulsifier. In certain embodiments, the basic composition contains from about 0.5 to about 30% emulsifier in the matrix, such as from about 1% to about 20%, and including from about 2 to about 10% emulsifier in the matrix. The final concentration will depend on the application of interest and balancing of other composition components.

All of the above percentages are percentages by weight.

### Composition Type B—Gum Based:

Alternatively, various commercially available gum and chewing gum bases can be utilized as a formulation substitute of the basic formulation. Gum bases can be from natural sources such as those derived from chicle (derived from the Manilkara chicle tree) or synthesized from organic monomers. Other natural latex gum options include leche, caspi, sorva, nispero, tunu, jelutong. Typical chewing gum bases contain constituents similar in ratios as the basic formulation described. Gum bases can include, but are not limited to: Alpha Global BB, Alpha Grande BB, Alpha Grande BB T, Gum Ball BB, Gum Ball BB T, Ladco BB, Ladco BB T, LG BB, LG BB T, Liberty BB, Liberty T BB, Magna BB 30280, Magna BB T 30515, Magna III BB, Magna IV BB, Magna T BB, Malibu BB, Valor BB, Valor BB T, Wicks BB T, Diamond Base, Dreyco Base, Dreyco Base T LC, Dreyfus Base 37199, Dreyfus Base T 37199, Dreyfus Base T 40981, Firm Paloja, Nova Base, Nova Base T, Nova Base T LC, Palohiloja T (LA Dreyfus Company) as well as various other commercially available types.

### Composition Type C—Hydrocarbon Based:

An alternate basic composition comprises a malleable hydrocarbon wax composition that is soft and putty or clay-like when deformed by hand. The composition can be contain feed-stock petroleum distillates, Typical suppliers include Exxon Mobile, International Group Inc., Clarus Specialty Products Inc., Accu-Blend Corporation, and other specialty manufacturers or formulators of wax-based products.

Relevant characteristics of a hydrocarbon wax based composition include that it is soft but maintains integrity at or around room temperature and during usage, that it does not shed layers or fragments during contact and use on a surface, that it encapsulates a contaminating medium as described in the composition above and the like.

Hydrocarbon wax-based compositions can be formulated and co-blended with constituents that provide the basic composition with the characteristics necessary to perform satisfactory solution-free and solvent-free surface cleaning with essential product features and qualities. Additives can include poly(isobutylene), oils, lubricants, colorants, fragrances, fillers, extenders, entrapping agents, and the like.

### Composition Type D—Silicone Based:

Another alternate basic composition comprises a malleable silicone-based composition that is also soft and putty or clay-like when deformed by hand. The composition can be formed from various ratios of poly-siloxanes and monomeric siloxanes. Monomeric and polymeric siloxanes can be obtained from commercial sources and chemical supply companies.

Similar to the compositions described above, relevant characteristics of a silicone-based composition include that it is soft but maintains integrity at or around room temperature and during usage, that it does not shed layers or fragments during contact and use on a surface, that it encapsulates a contaminating medium as described in the composition above and the like. Dimethicone, polydimethylsiloxane, silicone fluids, silicone emulsions, silicones with different viscosities, mixed silicon compositions and the like can be utilized.

Silicone-based compositions can be formulated and co-blended with constituents that provide the basic composition with the characteristics necessary to perform satisfactory solution-free and solvent-free surface cleaning with essential product features and qualities. Additives can include oils such as glycerin, lubricants, colorants, fragrances, fillers, extenders, entrapping agents, thixotrol and other alcohols, whitening agents, light-weight fillers, silica particulate and/or the like.

Monomeric siloxane can range from less than 1% to over 90%, such as from about 10 to about 80%, including from about 25 to about 75%. Polymeric siloxanes may range correspondingly form commensurate ratios from less than 1% to over 90%, such as from about 2% to about 50% and including from about 5 to about 30%. In one embodiment, the formulation is: 60% dimethyl siloxane, 20% silica, 10% thixotrol ST, 5% polydimethylsiloxane, 1% decamethyl cyclopentasiloxane, 1% glycerine, 1% glass micro-balloons as a lightweight filler.

### Poly(isobutylenes) Types:

Natural and synthetic rubber types can be utilized. Synthetic rubber types can be formed from isobutylene monomers. Iso-butylene monomers and corresponding polymers can include, but not limited to: 1,1-Dimethylethene, 1,1-Dimethylethylene, 1-Propene, 2-methyl-, 1-Propene, 2-methyl-, homopolymer, chlorinated, 115-11-7, 2-Methyl-1-propene, 2-Methylallyl radical, 2-Methylpropene, 2-Methylpropene-isobutylene, 2-Methylpropylene, 30000754, 68037-14-9, 9003-27-4, CCRIS 2281, CH2C(CH3)═CH2, EINECS 204-066-3, gamma-Butylene, HSDB 613, iso-C4H8, Isobutene, Isobutylene, Isobutylene see also Petroleum gases, liquefied [UN1055] [Flammable gas], isobutylene, various grades, isopropylidenemethylene, methylpropene, NISTC115117, NISTC15157956, polyisobutylene, chlorinated, Propene, 2-methyl-, sym-Dimethylethylene, UN 1055, UN 1075, UN1055, unsym. dimethylethylen, poly (isobutylene-b-indene-b-isobutylene), poly (isobutylene-co-4-methyl styrene), poly (isobutylene-alt maleic acid), and the like. Typical and representative polymers include, but are not limited to: poly-isobutylene, an isobutylene-isoprene copolymer, or styrene-butadiene copolymers.

### Emulsifier Types:

A variety of available emulsifiers can be utilized in the composition including mono-diglycerides emulsifiers including Sugin 471 PH 40, Sugin 471 PH 60, Sugin 471 PH 90; lactic esters of MG including Sugin 472 B I15; citric esters of MG including Sugin 472 C IKV and Sugin 472 C J; sorbitan esters including Sorbester 20, Sorbester 60, Sorbester 65, Sorbester 80; POE Sorbitan esters including Polysorbate 20, Polysorbate 60, Polysorbate 65, Polysorbate 80; polyglycerol esters including Sugin 475 HI, Sugin 476 M, specialty emulsfiers including Base HC 5, Base HC 9, Sugin SP 8019, Deca line, Del line, Tween (20) as well as a variety of other commercially available emulsifier types. Lecithins as emulsifiers can include, but are not limited to: Topcithin a standard fluid lecithins; Chocotop, lecithins for the chocolate industry, including selected liquid, fractionated, and compounded qualities; Emulfluid lecithins with fluid qualities with tailored emulsification profiles, including enzymatically hydrolyzed qualities as well as fractionated products; Emulpur and Emultop lecithins that are de-oiled qualities for a wide range of applications, Emultop being a hydrolyzed quality with superior emulsification power; Metarin—lecithins selected and/or fractionated de-oiled qualities as well as liquid compounds. Predominantly for “instant” applications and for applications requiring defined phospholipid patterns; Lecimulthin and Emulthin and the like. Lecithins can be derived from various sources including egg lecithin and soy lecithin. Lecithins can be obtained from both plant and animal raw materials. Other plant raw materials apart from the soybean include oilseeds such as rapeseed, sun flower seed and maize as well as other refined vegetable oils sources.

Likewise, various chelating agents can be employed to further facilitate entrapment and securing of various dirt and contaminating types within the matrix of the composition. U.S. Pat. No. 5,186,856 (BASF) describes a solvent free pre-treat based on a chelating agent which does not exhibit separation.

### Inert Filler Types:

Inert fillers that serve to increase composition volume and reduce cost include, but are not limited to: calcium carbonate, talc, weight reduction fillers such as light weight materials including Expancel™ 40, Expancel 80, and Expancel 120; dolomite; sand; diatomaceous earth; various plastic powders; oxides; metal powders; natural and inorganic powders; and the like.

Fillers can include, but are not limited to: calcium carbonate, weight lightening compounds such as Expancel™, aluminum hydrate, oxides, talc powder, sand, rubber powder, ethyl-vinyl acetate types (EVA), diatomaceous earth, sawdust and wood powder, borosilicate, fumed silicone, plastic powders and extenders, foaming agents, cellulose powder types, corn or other starch types, glass particles or filament types, graphite, paper powder and pulp types, and the like. The selection of a filler is predicated upon the filler not interfering, but rather either being either inert to the function of a composition or augmenting the intended activity of the composition.

Fillers can be added from in a range from 0.1% to over 90%. Usually, they will be added between a range from 1% to 75%. More usually, they will be added in a range between 5% and 60% and most often between 10% and 50%. Augmenting features of a filler can include that they help to reduce tack of a composition to a surface. Likewise, a filler can help to improve the consistency, texture, and/or feel of a composition. The selection of a filler type can help to reduce cost of a product by increasing the ration of low cost components in the composition.

### Surface Abrasives:

A wide range of different abrasives can be employed to facilitate contamination removal from a surface. The abrasive type and concentration can be adjusted to meet the requirements of a particular application or product type. Abrasives can include but are not limited to: pumice, silicon dioxide, fumed silicone, ground glass powder, ceramic powder, polishing powders used for metals or other surfaces, hard plastic powders, cellulose fiber types, inorganic salts, minerals, stone powders, and the like.

### Temperature Adjusted Activity:

In certain cases it may be desirable to utilize a temperature adjustment in the composition to modulate and improve its activity for adhering to and entrapping certain contamination types. For example it may be desirable to elevate the temperature of the composition so that the composition can more effectively entrap and entangle a species of contamination. The composition can be formulated to be more malleable or pliable so that it can more effectively conform to a surface. Likewise, at elevated temperatures, the composition may facilitate the loosening, release, or liberation of a contamination type from a surface because the contamination itself will be less adherent at an elevated temperature.

Alternatively, the composition can be formulated to function at below room temperature so that it can more effectively preserve a surface that it is being used to clean or decontaminate. Likewise, the composition can be formulated to facilitate its release at a lower temperature by lowering its adhesive properties. In the latter case, the formulation can be made more adhesive to a contamination type at an elevated temperature and less adhesive to a surface and thereby reducing its tack to a surface at reduced temperatures.

The composition can be formulated for activity in the temperature range from at or above 200° C. to −20° C., such as from between about 100° C. to −10° C., and including from between about 50° C. and about 0° C., e.g., from between about 30° C. and about 15° C. (ambient conditions).

Temperature activated polymers will find use for adjusting the optimal temperature setting and activity for the composition. By way of example, polycaprolactones, polyurethanes, hot melt compositions, ethyl-vinyl acetates (EVA), microcrystalline waxes, Interlemer™ polymer compositions (Landec Corporation), high and low temperature poly(isobutylenes) and the like may find use for temperature adjustment.

### Additional Additives:

Alternatively additives can be utilized to change or modify a property of the composition. By way of example, oils, lubricants, surfactants, waxes or the like can be added to a putty to reduce stickiness. Emulsifiers can be added to improve consistency and as an aid for mixing. Fragrances can be added to alter a smell sensation during use. Gum bases may be added to increase pliability. Encapsulating elements may be added to provide a carrier means for adding non-miscible materials.

The hydrophobic and hydrophilic nature can be adjusted in the composition to adjust and modulate the effectiveness of cleaning and removing commensurate contamination types from a surface. For example, hydrophobic hydrocarbons such as wax and petroleum jelly can be added to increase the hydrophobic nature of the composition. For example, degreasing agents can be added to the composition to improve the composition's ability to remove hydrophobic grease from a surface. Likewise, aqueous elements including water, alcohols esters, detergents or the like can be added to the composition to make it more hydrophilic.

Lubricants can be added to reduce the tack of the composition including, silicone oils, siloxanes, rubberizing compounds, polyethylene oxides, polysiloxanes waxes, liquid and powdered Teflon™ compositions and various other lubricating polymers. Lubricants can be added in at concentrations that will help reduce stick and tack on a surface of interest or to finger touch.

Natural and synthetic oils, mineral oils, softening agents, skin cream components, hand cream components, soothing agents and the like can be added for particular applications where it is useful to include dermatological care constituent that can aid and assist in leaving behind a desired layer of the agent on a surface. The composition can thereby be useful for cleansing a contamination from the surface as well as leaving behind a therapeutic film to assist in the care of the surface being cleansed.

Additional adhesives and glues can be added to the composition formulation to further increase it adherent characteristics to dirt and other surface contaminating species. A wide range of different adhesives typically used in pressure sensitive labels, tapes, adherent note pads, stickers, industrial adhesives, glues, and the like may find use as additives to the composition.

Surfactants for Promoting Transport from Surface to Composition:

The surfactant constituent can comprise one or more non-ionic, anionic, cationic, zwitterionic or natural surfactants or mixtures thereof. Examples of anionic surfactants which may be used in the surfactant constituent include alcohol sulfates and sulfonates, alcohol phosphates and phosphonates, alkyl ester sulfates, alkyl diphenyl ether sulfonates, alkyl sulfates, alkyl ether sulfates, sulfate esters of an alkyphenoxy polyoxyethylene ethanol, alkyl monoglyceride sulfates, alkyl sulfonates, alkyl ether sulfates, alpha-olefin sulfonates, beta-alkoxy alkane sulfonates, alkyl ether sulfonates, ethoxylated alkyl sulfonates, alkylaryl sulfonates, alkylaryl sulfates, alkyl monoglyceride sulfonates, alkyl carboxylates, alkyl ether carboxylates, alkyl alkoxy carboxylates having 1 to 5 moles of ethylene oxide, alkylpolyglycolethersulfates (containing up to 10 moles of ethylene oxide), sulfosuccinates, octoxynol or nonoxynol phosphates, taurates, fatty taurides, fatty acid amide polyoxyethylene sulfates, acyl glycerol sulfonates, fatty oleyl glycerol sulfates, alkyl phenol ethylene oxide ether sulfates, paraffin sulfonates, alkyl phosphates, isethionates, N-acyl taurates, alkyl succinamates and sulfosuccinates, alkylpolysaccharide sulfates, alkylpolyglucoside sulfates, alkyl polyethoxy carboxylates, and sarcosinates or mixtures thereof. These anionic surfactants may be provided as salts with one or more organic counterions, e.g., ammonium, or inorganic counterions, especially as salts of one or more alkaline earth or alkaline earth metals, e.g., sodium.

Further examples of anionic surfactants include water soluble salts or acids of the formula (ROSO₃)ₓM or (RSO₃)ₓM wherein R is preferably a C₆-C₂₄ hydrocarbyl, preferably an alkyl or hydroxyalkyl having a C₁₀-C₂₀ alkyl component, more preferably a C₁₂-C₁₈ alkyl or hydroxyalkyl, and M is H or a mono-, di- or tri-valent cation, e.g., an alkali metal cation (e.g., sodium, potassium, lithium), or ammonium or substituted ammonium (e.g., methyl-, dimethyl-, and trimethyl ammonium cations and quaternary ammonium cations, such as tetramethyl-ammonium and dimethyl piperdinium cations and quaternary ammonium cations derived from alkylamines such as ethylamine, diethylamine, triethylamine, and mixtures thereof, and the like) and x is an integer, preferably 1 to 3, most preferably 1. Materials sold under the Hostapur and Biosoft trademarks are examples of such anionic surfactants. Still further examples of anionic surfactants include alkyl-diphenyl-ethersulphonates and alkyl-carboxylates.

Also useful in the anionic surfactant constituent are diphenyl disulfonates, and salt forms thereof, such as a sodium salt of diphenyl disulfonate commercially available as DOWFAX 3B2. Such diphenyl disulfonates are included in certain preferred embodiments of the invention in that they provide not only a useful cleaning benefit but concurrently also provide a useful degree of hydrotropic functionality. Other anionic surfactants can include salts (including, for example, sodium, potassium, ammonium, and substituted ammonium salts such as mono-, di-and triethanolamine salts) of soap, C₆-C₂₀ linear alkylbenzenesulfonates, C₆-C₂₂ primary or secondary alkanesulfonates, C₆-C₂₄ olefinsulfonates, sulfonated polycarboxylic acids prepared by sulfonation of the pyrolyzed product of alkaline earth metal citrates, C₆-C₂₄ alkylpolyglycolethersulfates, alkyl ester sulfates such as C₁₄-C₁₆ methyl ester sulfates; acyl glycerol sulfonates, fatty oleyl glycerol sulfates, alkyl phenol ethylene oxide ether sulfates, paraffin sulfonates, alkyl phosphates, isethionates such as the acyl isethionates,N-acyl taurates, alkyl succinamates and sulfosuccinates, monoesters of sulfosuccinate (especially saturated and unsaturated C₁₂-C₁₈ monoesters) diesters of sulfosuccinate (especially saturated and unsaturated C₆-C₁₄ diesters), acyl sarcosinates, sulfates of alkylpolysaccharides such as the sulfates of alkylpolyglucoside, branched primary alkyl sulfates, alkyl polyethoxy carboxylates such as those of the formula RO(CH₂CH₂O)ₖCH₂COO⁻M⁺ wherein R is a C₈-C₂₂ alkyl, k is an integer from 0 to 10, and M is a soluble salt-forming cation. Examples of the foregoing anionic surfactants are available under the following tradenames: RHODAPON, STEPANOL, SURFINE, SANDOPAN, NEODOX, BIOSOFT, and AVANEL. A preferred anionic surfactant is sodium laurethyl sulfonate.

Examples of nonionic surfactants which may be used in the surfactant constituent include polyethylene oxide condensates of alkyl phenols. These compounds include the condensation products of alkyl phenols having an alkyl group containing from about 6 to 12 carbon atoms in either a straight chain or branched chain configuration with ethylene oxide, the ethylene oxide being present in an amount equal to 5 to 25 moles of ethylene oxide per mole of alkyl phenol. The alkyl substituent in such compounds can be derived, for example, from polymerized propylene, diisobutylene and the like. Examples of compounds of this type include nonyl phenol condensed with about 9.5 moles of ethylene oxide per mole of nonyl phenol; dodecylphenol condensed with about 12 moles of ethylene oxide per mole of phenol; dinonyl. phenol condensed with about 15 moles of ethylene oxide per mole of phenol and diisooctyl phenol condensed with about 15 moles of ethylene oxide per mole of phenol.

Further useful nonionic surfactants include the condensation products of aliphatic alcohols with from about 1 to about 60 moles of ethylene oxide. The alkyl chain of the aliphatic alcohol can either be straight or branched, primary or secondary, and generally contains from about 8 to about 22 carbon atoms. Examples of such ethoxylated alcohols include the condensation product of myristyl alcohol condensed with about 10 moles of ethylene oxide per mole of alcohol and the condensation product of about 9 moles of ethylene oxide with coconut alcohol (a mixture of fatty alcohols with alkyl chains varying in length from about 10 to 14 carbon atoms). Other examples are those C₆-C₁₁ straight-chain alcohols which are ethoxylated with from about 3 to about 6 moles of ethylene oxide. Their derivation is well known in the art. Examples include Alfonic® 810-4.5 (also available as Teric G9A5), which is described in product literature from Sasol as a C₈-C₁₀ having an average molecular weight of 356, an ethylene oxide content of about 4.85 moles (about 60 wt. %), and an HLB of about 12; Alfonic® 810-2, which is described in product literature from Sasol as a C₈-C₁₀ having an average molecular weight of 242, an ethylene oxide content of about 2.1 moles (about 40 wt. %), and an HLB of about 12; and Alfonic® 610-3.5, which is described in product literature from Sasol as having an average molecular weight of 276, an ethylene oxide content of about 3.1 moles (about 50 wt. %), and an HLB of 10. Product literature from Sasol also identifies that the numbers in the alcohol ethoxylate name designate the carbon chain length (numbers before the hyphen) and the average moles of ethylene oxide (numbers after the hyphen) in the product.

Further exemplary useful nonionic surfactants include alcohol ethoxylates including C₁₀ oxo-alcohol ethoxylates available from BASF under the Lutensol ON tradename. They are available in grades containing from about 3 to about 11 moles of ethylene oxide (available under the names Lutensol ON 30; Lutensol ON 50; Lutensol ON 60; Lutensol ON 65; Lutensol ON 66; Lutensol ON 70; Lutensol ON 80; and Lutensol ON 110). Yet further examples of ethoxylated alcohols include the Neodol® 91 series non-ionic surfactants available from Shell Chemical Company which are described as C₉-C₁₁ ethoxylated alcohols. The Neodol® 91 series non-ionic surfactants of interest include Neodol 91-2.5, Neodol 91-6, and Neodol 91-8. Neodol 91-2.5 has been described as having about 2.5 ethoxy groups per molecule; Neodol 91-6 has been described as having about 6 ethoxy groups per molecule; and Neodol 91-8 has been described as having about 8 ethoxy groups per molecule. Still further examples of ethoxylated alcohols include the Rhodasurf® DA series non-ionic surfactants available from Rhodia which are described to be branched isodecyl alcohol ethoxylates. Rhodasurf DA-530 has been described as having 4 moles of ethoxylation and an HLB of 10.5; Rhodasurf DA-630 has been described as having 6 moles of ethoxylation with an HLB of 12.5; and Rhodasurf DA-639 is a 90% solution of DA-630.

Further examples of ethoxylated alcohols include those from Tomah Products (Milton, Wis.) under the Tomadol tradename with the formula RO(CH₂CH₂O)ₙH where R is the primary linear alcohol and n is the total number of moles of ethylene oxide. The ethoxylated alcohol series from Tomah include 91-2.5; 91-6; 91-8—where R is linear C9/C10/C11 and n is 2.5,6, or 8; 1-3; 1-5; 1-7; 1-73B; 1-9;—where R is linear C11 and n is 3, 5, 7 or 9; 23-1; 23-3; 23-5; 23-6.5—where R is linear C12/C13 and n is 1,3, 5, or 6.5; 25-3; 25-7; 25-9; 25-12—where R is linear C12/C13 C14/C15 and n is 3, 7, 9, or 12; and 45-7; 45-13—where R is linear C14/C15 and n is 7 or 13.

Other examples of nonionic surfactants include primary and secondary linear and branched alcohol ethoxylates, such as those based on C₆-C₁₈ alcohols which further include an average of from 2 to 80 moles of ethoxylation per mol of alcohol. These examples include the Genapol UD series from Clariant, described as tradenames Genapol UD 030, C₁₁-Oxo-alcohol polyglycol ether with 3 EO; Genapol UD, 050 C₁₁-Oxo-alcohol polyglycol ether with 5 EO; Genapol UD 070, C₁₁-Oxo-alcohol polyglycol ether with 7 BO; Genapol UD 080, C₁₁-Oxo-alcohol polyglycol ether with 8 EO; Genapol UD 088, C₁₁-Oxo-alcohol polyglycol ether with 8 EO; and Genapol UD 110, C₁₁-Oxo-alcohol polyglycol ether with 11 EO.

Other examples of useful nonionic surfactants include those having a formula RO(CH₂CH₂O)ₙH wherein R is a mixture of linear, even carbon-number hydrocarbon chains ranging from C₁₂H₂₅ to C₁₆H₃₃ and n represents the number of repeating units and is a number of from about 1 to about 12. Surfactants of this formula are presently marketed under the Genapol® tradename, available from Clariant, Charlotte, N.C., include the 26-L series of the general formula RO(CH₂CH₂O)ₙH wherein R is a mixture of linear, even carbon-number hydrocarbon chains ranging from C₁₂H₂₅ to C₁₆H₃₃ and n represents the number of repeating units and is a number of from 1 to about 12, such as 26-L-1,26-L-1.6, 26-L-2, 26-L-3, 26-L-5, 26-L-45, 26-L-50, 26-L-60, 26-L-60N, 26-L-75, 26-L-80, 26-L-98N, and the 24-L series, derived from synthetic sources and typically contain about 55% C₁₂ and 45% C₁₄ alcohols, such as 24-L-3, 24-L-45, 24-L-50, 24-L-60, 24-L-60N, 24-L-75, 24-L-92, and 24-L-98N. From product literature, the single number following the “L” corresponds to the average degree of ethoxylation (numbers between 1 and 5) and the two digit number following the letter “L” corresponds to the cloud point in ° C. of a 1.0 wt. % solution in water.

A further class of nonionic surfactants which are contemplated to be useful include those based on alkoxy block copolymers, and in particular, compounds based on ethoxy/propoxy block copolymers. Polymeric alkylene oxide block copolymers include nonionic surfactants in which the major portion of the molecule is made up of block polymeric C₂₋C₄ alkylene oxides. Such nonionic surfactants, while preferably built up from an alkylene oxide chain starting group, and can have as a starting nucleus almost any active hydrogen containing group including, without limitation, amides, phenols, thiols and secondary alcohols.

One group of such useful nonionic surfactants containing the characteristic alkylene oxide blocks are those which may be generally represented by the formula (A): HO-(EO)ₓ(PO)_{y}(EO)_{z}—H (A) where EO represents ethylene oxide, PO represents propylene oxide, y equals at least 15, (EO)_{x+y} equals 20 to 50% of the total weight of said compounds, and, the total molecular weight is preferably in the range of about 2000 to 15,000. These 5 surfactants are available under the PLURONIC tradename from BASF or Emulgen from Kao. Another group of nonionic surfactants appropriate for use in the new compositions can be represented by the formula (B): R-(EO,PO)ₐ(EO,PO)_{b}—H (B) wherein R is an alkyl, aryl or aralkyl group, where the R group contains 1 to 20 carbon atoms, the weight percent of EO is within the range of 0 to 45% in one of the blocks a, b, and within the range of 60 to 100% in the other of the blocks a, b, and the total number of moles of combined EO and PO is in the range of 6 to 125 moles, with 1 to 50 moles in the PO rich block and 5 to 100 moles in the EO rich block.

Further nonionic surfactants which in general are encompassed by Formula B include butoxy derivatives of propylene oxide/ethylene oxide block polymers having molecular weights within the range of about 2000-5000. Still further Useful nonionic surfactants containing polymeric butoxy (BO) groups can be represented by formula (C) as follows: RO—(BO)ₙ(EO)ₓ—H (C) wherein R is an alkyl group containing 1 to 20 carbon atoms, n is about 5-15 and x is about 5-15.

Also useful as the nonionic block copolymer surfactants, which also include polymeric butoxy groups, are those which may be represented by the following formula (D): HO-(EO)_{X}(BO)ₙ(EO)_{y}—H (D) wherein n is about 5-15, preferably about 15, x is about 5-15, preferably about 15, and y is about 5-15, preferably about 15.

Still further useful nonionic block copolymer surfactants include ethoxylated derivatives of propoxylated ethylene diamine, which may be represented by the following formula:

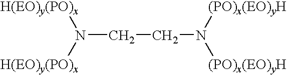

where (EO) represents ethoxy, (PO) represents propoxy, the amount of (PO)ₓ is such as to provide a molecular weight prior to ethoxylation of about 300 to 7500, and the amount of (EO) is such as to provide about 20% to 90% of the total weight of said compound.

Surfactants based on amine oxides are also contemplated to be useful in the surfactant constituent in the present inventive compositions. Exemplary amine oxides include: alkyl di(C₁-C₇) amine oxides in which the alkyl group has about 10-20, and preferably 12-16 carbon atoms, and can be straight or branched chain, saturated or unsaturated. Examples of such compounds include lauryl dimethyl amine oxide, myristyl dimethyl amine oxide, and those in which the alkyl group is a mixture of different amine oxide, dimethyl cocoamine oxide, dimethyl (hydrogenated tallow) amine oxide, and myristyl/palmityl dimethyl amine oxide; alkyl di(hydroxyl) C₁-C₇ amine oxides in which the alkyl group has about 10-20, and preferably 12-16 carbon atoms, and can be straight or branched chain, saturated or unsaturated. Examples of such compounds include bis(2-hydroxyethyl) cocoamine oxide, bis(2-hydroxyethyl) tallowamine oxide; and bis(2-hydroxyethyl) stearylamine oxide; alkylamidopropyl di(C₁-C₇)amine oxides in which the alkyl group has about 10-20, and preferably 12-16 carbon atoms, and can be straight or branched chain, saturated or unsaturated. Examples of such compounds include cocoamidopropyl dimethyl amine oxide and tallowamidopropyl dimethyl amine oxide; and alkylmorpholine oxides in which the alkyl group has about 10-20, and preferably 12-16 carbon atoms, and can be straight or branched chain, saturated or unsaturated. Further classes of surfactants which are contemplated as being useful in the cosurfactant constituent include alkylmonoglyocosides and alkylpolyglycosides which include known nonionic surfactants which are alkaline and electrolyte stable.

Alkylmonoglycosides and alkylpolyglycosides are prepared generally by reacting a monosaccharide, or a compound hydrolyzable to a monosaccharide with an alcohol such as a fatty alcohol in an acid medium. Various glycoside and polyglycoside compounds including alkoxylated glycosides and processes for making them are disclosed in U.S. Pat. Nos. 2,974,134; 3,219,656; 3,598,865; 3,640,998; 3,707,535, 3,772,269; 3,839,318; 3,974,138; 4,223,129 and 4,528,106. One exemplary group of such useful alkylpolyglycosides include those according to the formula: R₂O—(CₙH₂ₙO)ᵣ—(Z)ₓ wherein: R₂ is a hydrophobic group selected from alkyl groups, alkylphenyl groups, hydroxyalkylphenyl groups as well as mixtures thereof, wherein the alkyl groups may be straight chained or branched, and which contain from about 8 to about 18 carbon atoms, n has a value of 2-8, especially a value of 2 or 3; r is an integer from 0 to 10, but is preferably 0, Z is derived from glucose; and, x is a value from about 1 to 8, preferably from about 1.5 to 5. Preferably the alkylpolyglycosides are nonionic fatty alkylpolyglucosides which contain a straight chain or branched chain C₈-C₁₅ alkyl group, and have an average of from about 1 to 5 glucose units per fatty alkylpolyglucoside molecule. More preferably, the nonionic fatty alkylpolyglucosides which contain straight chain or branched C₈-C₁₅ alkyl group, and have an average of from about 1 to about 2 glucose units per fatty alkylpolyglucoside molecule. Exemplary useful include, for example APG 325 CS Glycoside® which is described as being a 50% C₉-C₁₁ alkyl polyglycoside, also commonly referred to as D-glucopyranoside, (commercially available from Henkel KGaA) and Glucopon® 625 CS which is described as being a 50% C₁₀-C₁₆ alkyl polyglycoside, also commonly referred to as a D-glucopyranoside, (ex. Henkel).

By way of non-limiting example exemplary amphoteric surfactants, also known as zwitterionic surfactants, which are contemplated to be useful in the surfactant constituent include one or more water-soluble sulfobetaine surfactants such as sulfobetaine-12 and betaine surfactants which may be represented by the general formula:

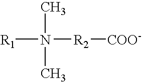

wherein R₁ is an alkyl group containing from 8 to 18 carbon atoms, or the amido radical which may be represented by the following general formula:

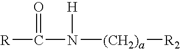

wherein R is an alkyl group having from 8 to 18 carbon atoms, a is an integer having a value of from 1 to 4 inclusive, and R₂ is a C₁-C₄ alkylene group. Examples of such water-soluble betaine surfactants include dodecyl dimethyl betaine, as well as cocoamidopropylbetaine.

Surfactants based on amine oxides are also contemplated to be useful in the surfactant constituent in the present inventive compositions. Exemplary amine oxides include: alkyl di(C₁-C₇) amine oxides in which the alkyl group has about 10-20, and preferably 12-16 carbon atoms, and can be straight or branched chain, saturated or unsaturated. Examples of such compounds include lauryl dimethyl amine oxide, myristyl dimethyl amine oxide, and those in which the alkyl group is a mixture of different amine oxide, dimethyl cocoamine oxide, dimethyl (hydrogenated tallow) amine oxide, and myristyl/palmityl dimethyl amine oxide; alkyl di(hydroxy C₁-C₇)amine oxides in which the alkyl group has about 10-20, and preferably 12-16 carbon atoms, and can be straight or branched chain, saturated or unsaturated. Examples of such compounds include bis(2-hydroxyethyl) cocoamine oxide, bis(2-hydroxyethyl) tallowamine oxide; and bis(2-hydroxyethyl) stearylamine oxide; alkylamidopropyl di(C₁-C₇)amine oxides in which the alkyl group has about 10-20, and preferably 12-16 carbon atoms, and can be straight or branched chain, saturated or unsaturated. Examples of such compounds include cocoamidopropyl dimethyl amine oxide and tallowamidopropyl dimethyl amine oxide; and alkylmorpholine oxides in which the alkyl group has about 10-20, and preferably 12-16 carbon atoms, and can be straight or branched chain, saturated or unsaturated.

Various stain remover systems examples are described in U.S. Pat. Nos. 5,891,197, 5,872,090, 5,849,039, 5,789,368 and 5,681,355, 7,047,582 and U.S. patent application Ser. No. 60/190,640. Typically the stain remover system comprises a stain removal composition as well as an absorbent stain receiver article.

### Germicidal/Disinfectant Additives:

Particularly preferred for use as the cationic surfactant having germicidal properties are those cationic surfactants which are found to provide a broad antibacterial or sanitizing function. Any cationic surfactant which satisfies these requirements may be used and are considered to be within the scope of the present invention, and mixtures of two or more cationic surface active agents, viz., cationic surfactants may also be used. Cationic surfactants are well known, and useful cationic surfactants may be one or more of those described for example in McCutcheon's Functional Materials, Vol. 2, 1998;. Kirk-Othmer, Encyclopaedia of Chemical Technology, 4th Ed., Vol. 23, pp. 478-541 (1997. These are also described in the respective product specifications and literature available from the suppliers of these cationic surfactants.

Examples of preferred cationic surfactant compositions useful in the practice of the instant invention are those that provide a germicidal effect to the concentrate compositions, and especially preferred are quaternary ammonium compounds and salts thereof, which may be characterized by the general structural formula:

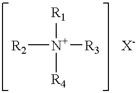

where at least one of R₁, R₂, R₃ and R₄ is a alkyl, aryl or alkylaryl substituent of from 6 to 26 carbon atoms, and the entire cation portion of the molecule has a molecular weight of at least 165. The alkyl substituents may be long-chain alkyl, long-chain alkoxyaryl, long-chain alkylaryl, halogen-substituted long-chain alkylaryl, long-chain alkylphenoxyalkyl, arylalkyl, etc. The remaining substituents on the nitrogen atoms other than the above mentioned alkyl substituents are hydrocarbons usually containing no more than 12 carbon atoms. The substituents R₁, R₂, R₃ and R₄ may be straight-chained, or may be branched, but are preferably straight-chained, and may include one or more amide, ether or ester linkages. The counter-ion X may be any salt-forming anion that permits water solubility of the quaternary ammonium complex.

Exemplary quaternary ammonium salts within the above description include the alkyl ammonium halides such as cetyl trimethyl ammonium bromide, alkyl aryl ammonium halides such as octadecyl dimethyl benzyl ammonium bromide, N-alkyl pyridinium halides such as N-cetyl pyridinium bromide. Other suitable types of quaternary ammonium salts include those in which the molecule contains either amide, ether or ester linkages such as octyl phenoxy ethoxy ethyl dimethyl benzyl ammonium chloride and N-(laurylcocoaminoformylmethyl)-pyridinium chloride. Other very effective types of quaternary ammonium compounds which are useful as germicides include those in which the hydrophobic radical is characterised by a substituted aromatic nucleus as in the case of lauryloxyphenyltrimethyl ammonium chloride, cetylaminophenyltrimethyl ammonium methosulfate, dodecylphenyltrimethyl ammonium methosulfate, dodecylbenzyltrimethyl ammonium chloride and chlorinated dodecylbenzyltrimethyl ammonium chloride.

Preferred quaternary ammonium compounds which act as germicides and which are to be found useful in the practice of the present invention include those that have the structural formula:

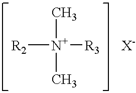

wherein R₂ and R₃ are the same or different C₈-C₁₂alkyl, or R₂ is C₁₂₋₁₆alkyl, C₈₋₁₈alkylethoxy, C₈₋₁₈alkylphenoxyethoxy and R₃ is benzyl, and X is a halide, for example chloride, bromide or iodide, or is a methosulfate anion. The alkyl groups recited in R₂ and R₃ may be straight-chained or branched, but are preferably substantially linear.

Particularly useful quaternary germicides include compositions which include a single quaternary compound, as well as mixtures of two or more different quaternary compounds. Such useful quaternary compounds are available under the BARDAC®, BARQUAT®, HYAMINE®, LONZABAC®, and ONYXIDE® trademarks, which are more fully described in, for example, McCutcheon's Functional Materials (Vol. 2), North American Edition, 1998, as well as the respective product literature from the suppliers identified below. For example, BARDAC® 205M is described to be a liquid containing alkyl dimethyl benzyl ammonium chloride, octyl decyl dimethyl ammonium chloride; didecyl dimethyl ammonium chloride, and dioctyl dimethyl ammonium chloride (50% active) (also available as 80% active (BARDAC® 208M)); described generally in McCutcheon's as a combination of alkyl dimethyl benzyl ammonium chloride and dialkyl dimethyl ammonium chloride); BARDAC® 2050 is described to be a combination of octyl decyl dimethyl ammonium chloride/didecyl dimethyl ammonium chloride, and dioctyl dimethyl ammonium chloride (50% active) (also available as 80% active (BARDAC® 2080)); BARDAC® 2250 is described to be didecyl dimethyl ammonium chloride (50% active); BARDAC® LF (or BARDAC® LF-80), described as being based on dioctyl dimethyl ammonium chloride (BARQUAT® MB-50, MX-50, OJ-50 (each 50% liquid) and MB-80 or MX-80 (each 80% liquid) are each described as an alkyl dimethyl benzyl ammonium chloride; BARDAC® 4250 and BARQUAT® 4250Z (each 50% active) or BARQUAT® 4280 and BARQUAT® 4280Z (each 80% active) are each described as alkyl dimethyl benzyl ammonium chloride/alkyl dimethyl ethyl benzyl ammonium chloride. Also, HYAMINE® 1622, described as diisobutyl phenoxy ethoxy ethyl dimethyl benzyl ammonium chloride (50% solution); HYAMINE® 3500 (50% actives), described as alkyl dimethyl benzyl ammonium chloride (also available as 80% active (HYAMINE® 3500-80)); and HYMAINE® 2389 described as being based on methyldodecylbenzyl ammonium chloride and/or methyldodecylxylene-bis-trimethyl ammonium chloride. (BARDAC®, BARQUAT® and HYAMINE® are presently commercially available from Lonza, Inc. Fairlawn, N.J.). BTC® 50 NF (or BTC® 65 NF) is described to be alkyl dimethyl benzyl ammonium chloride (50% active); BTC® 99 is described as didecyl dimethyl ammonium chloride (50% active); BTC® 776 is described to be myrisalkonium chloride (50% active); BTC® 818 is described as being octyl decyl dimethyl ammonium chloride, didecyl dimethyl ammonium chloride, and dioctyl dimethyl ammonium chloride (50% active) (available also as 80% active (BTC® 818-80%)); BTC® 824 and BTC® 835 are each described as being of alkyl dimethyl benzyl ammonium chloride (each 50% active); BTC® 885 is described as a combination of BTC® 835 and BTC® 818 (50% active) (available also as 80% active (BTC® 888)); BTC® 1010 is described as didecyl dimethyl ammonium chlorides (50% active) (also available as 80% active (BTC® 1010-80)); BTC® 2125 (or BTC® 2125 M) is described as alkyl dimethyl benzyl ammonium chloride and alkyl dimethyl ethylbenzyl ammonium chloride (each 50% active) (also available as 80% active (BTC® 2125 80 or BTC® 2125 M)); BTC® 2565 is described as alkyl dimethyl benzyl ammonium chloride (50% active) (also available as 80% active (BTC® 2568)); BTC® 8248 (or BTC® 8358) is described as alkyl dimethyl benzyl ammonium chloride (80% active) (also available as 90% active (BTC® 8249)); ONYXIDE® 3300 is described as n-alkyl dimethyl benzyl ammonium saccharinate (95% active). (BTC® and ONYXIDE® are presently commercially available from Stepan Company, Northfield, Ill.) Polymeric quaternary ammonium salts based on these monomeric structures are also considered desirable for the present invention. One example is POLYQUAT®, described as being a 2-butenyldimethyl ammonium chloride polymer. Pluronic surfactants can include by way of example, F-68.

Various other germicidal and antibacterial agents and disinfectants can be added to the composition to ensure that bacterial or viral growth does not occur with in the composition matrix and whereby the matrix effectively kills all infective organisms that it comes in contact with. Furthermore, the composition can be formulated to deposit a metered amount of germicidal or antibacterial agent on the surface that it comes in contact with where by leaving an antibacterial or antiviral layer on the surface that further disinfects the surface being cleansed.

### Common Colorants and Pigments:

Fluorescent dyes and pigments can find use in various product applications and mediums and formats to improve the coloration of the initial product as well as acting to create a strong contrast in the composition matrix indicating that a contaminating species has been transferred into the matrix. Fluorescent dye compounds can include, but are not limited to: fluorescein, fluorescein, resourcinolphthalein, rhodamine, imidazolium cations, pyridoimidazolium cations, dinitrophenyl, tetramethylrhodamine and the like. A wide range of fluorescent dyes that can be activated at various wavelengths and emit light at lower wavelengths can be purchased from Dayglo Inc., Swada Chemical, Sigma-Aldrich (Saint Louis, Mo.) or Molecular Probes (Eugene, Oregon).

### Thermochromic Colorant Additives for Activity Indication:

Thermochromic dyes and colorants can be added to the composition formulation to serve as an indicating means to show that a particular composition has been temperature activated for optimal use. Typically, a composition may be initially firm prior to use and in its intended package. The firm composition can be activated for optimal use by softening it by hand. Kneading and softening can raise the temperature of the composition to improve its elasticity and pliability. The softening process can be indicated by including an thermally sensitive dye in the composition that changes color at an optimal temperature for use.

Thermochromic dyes can find use in a variety of compositions and applications and formats. Thermochromic dyes can include but are not limited to compounds including: bis(2-amino-4-oxo-6-methylpyrimidinium)-tetrachlorocuprate(II); bis(2-amino-4-chloro-6-methylpyrimidinium) hexachlorod-icuprate(II); cobalt chloride; 3,5-dinitro salicylic acid; leuco dyes; spiropyrenes, bis(2-amino-4-oxo-6-methylpyrimidinium) tetrachlorocuprate(II) and bis(2-amino-4-chloro-6-methylpyrimidinium) hexachlorodicuprate(II), benzo- and naphthopyrans (Chromenes), poly(xylylviologen dibromide, di-beta-naphthospiropyran, Ferrocene-modified bis(spiropyridopyran), isomers of 1-isopropylidene-2-[1-(2-methyl-5-phenyl-3-thienyl)ethylidene]-succinic anhydride and the Photoproduct 7,7adihydro-4,7,7,7a-tetramethyl-2-phenylbenzo[b]thiophene-5,6-dicarboxylic anhydride, and the like.

Other thermochromic dyes of interest include leucodyes including color to colorless and color to color formations, vinylphenylmethane-leucocynides and derivatives, fluoran dyes and derivatives, thermochromic pigments, micro and nano-pigments, molybdenum compounds, doped or undoped vanadium dioxide, indolinospirochromenes, melting waxes, encapsulated dyes, liquid crystalline materials, cholesteric liquid crystalline materials, spiropyrans, polybithiophenes, bipyridine materials, microencapsulated, mercury chloride dyes, tin complexes, combination thermochromic/photochromic materials, heat formable materials which change structure based on temperature, natural thermochromic materials such as pigments in beans, various thermochromic inks sold by Securink Corp. (Springfield, Va.), Matusui Corp., Liquid Crystal Research Crop., or any acceptable thermochromic materials with the capacity to report a temperature change or can be photo-stimulated and the like. The chromic change agent selected will depend on a number of factors including cost, material loading, color change desired, levels or color hue change, reversibility or irreversibility, stability, and the like.

Alternative thermochromic materials can be utilized including, but not limited to: light-induced metastable state in a thermochromic copper (II) complex Chem. Commun., 2002, (15), 1578-1579 under goes a color change from red to purple for a thermochromic complex, [Cu(dieten)2](BF4)2 (dieten=N,N-diethylethylenediamine); encapsulated pigmented materials from Omega Engineering Inc.; bis(2-amino-4-oxo-6-methyl-pyrimidinium)-tetrachlorocuprate(II); bis(2-amino-4-chloro-6-methylpyrimidinium)hexachlorod-icuprate(II); cobalt chloride; 3,5-dinitro salicylic acid; leuco dyes; spiropyrenes, bis(2-amino-4-oxo-6-methylpyrimidinium)-tetrachlorocuprate(II); bis(2-amino-4-chloro-6-methylpyrimidinium) hexachlorod-icuprate(II); cobalt chloride; 3,5-dinitro salicylic acid; leuco dyes; spiropyrenes, bis(2-amino-4-oxo-6-methylpyrimidinium) tetrachlorocuprate(II) and bis(2-amino-4-chloro-6-methylpyrimidinium) hexachlorodicuprate(II), benzo- and naphthopyrans (Chromenes), poly(xylylviologen dibromide, di-beta-naphthospiropyran, Ferrocene-modified bis(spiropyridopyran), isomers of 1-isopropylidene-2-[1-(2-methyl-5-phenyl-3-thienyl)ethylidene]-succinic anhydride and the Photoproduct 7,7adihydro-4,7,7,7a-tetramethyl-2-phenylbenzo[b]thiophene-5,6-dicarboxylic anhydride, and the like. Encapsulated leuco dyes are of interest since they can be easily processed in a variety of formats into a plastic or putty matrix. Liquid crystal materials can be conveniently applied as paints or inks to surfaces of color/shape/memory composites.

Thermochromic color to colorless options can include by way of example, but not by limitation: yellow to colorless, orange to color less, red to colorless, pink to colorless, magenta to colorless, purple to colorless, blue to colorless, turquoise to colorless, green to colorless, brown to colorless, black to colorless. Color to color options include but are not limited to: orange to yellow, orange to pink, orange to very light green, orange to peach; red to yellow, red to orange, red to pink, red to light green, red to peach; magenta to yellow, magenta to orange, magenta to pink, magenta to light green, magenta to light blue; purple to red, purple to pink, purple to blue; blue to pink; blue to light green, dark blue to light yellow, dark blue to light green, dark blue to light blue; turquoise to light green, turquoise to light blue, turquoise to light yellow, turquoise to light peach, turquoise to light pink; green to yellow, dark green to orange, dark green to light green, dark green to light pink; brown and black to a variety of assorted colors, and the like. Colors can be deeply enriched using fluorescent and glow-in-the-dark or photo-luthinescent pigments as well as related color additives.

Reversible and irreversible versions of the color change agent can be employed depending on the desired embodiment of interest. Reversible agents can be employed where it is desirable to have a multi-use effect or reuse the color change effect. For example, products with continued and repeated use value will find utility of a reversible color change component comprising the final embodiment. In this case it would be desirable to utilize a reversible thermochromic or luminescent material which can be repeated during usage. In another example, it may be desirable to record a single color change permanently. In this case, it would be desirable to utilize a thermochromically irreversible material which changes from one color to another giving rise to a permanent change and indicating that the composition should be discarded after use.

Thermochromic dyes can be added in from 0.001% by weight to 80% by weight. More usually, the dye additive will find use in the range form 0.01% to 50% by weight. Usually, the additive will range in concentrations from 0.1% to 25%. Typically and most often, the colorant will be added in a range from 0.5% to 5%.

### Photochromic and Photoluminescent Dye Additives:

Photochromic dyes can find use in a variety of color change mediums and formats. Photochromic materials can include but are not limited to dyes including: 1,3-Dihydro-1,3,3-trimethylspiro[2H-indole-2,3′-[3H]phenanthr[9,10-b](1,4)oxazine]; bicyclo[2.2.1]hepta-2,5-diene; benzyl viologen dichloride; 4,4′-bipyridyl; 6-bromo-1′,3′-dihydro-1′,3′,3′-trimethyl-8-nitro spiro[2H; 5-chloro-1,3-dihydro-1,3,3-trimethylspiro[2H-indole-2,3′-(3H)naphth[2,1-b](1,4)oxazine]; 6,8-dibromo-1′,3′-dihydro-1′,3′,3′-trimethylspiro[2H; 1,1′-diheptyl-4,4′-bipyridinium dibromide; 1′,3′-dihydro-5′-methoxy-1′,3′,3′; 1′,3′-dihydro-8-methoxy-1′,3′3′-trimethyl-6-nitrospiro[2H]; 1′,3′-dihydro-1′3′,3′-trimethyl-6-nitrospiro[2H-1-benzopyran-2,2′-(2H)-indole]; 1,3-dihydro-1,3,3-trimethylspiro[2H-Indole-2,3′-[3H]naphth[2,1-b][1,4]oxazine]; 1,1′-dimethyl-4,4′-bipyridinium dichloride; 5-chloro-1,3-Dihydro-1,3,3-trimethylspiro[2H-indole-2,3′-(3H)phenanthr[9,10-b](1,4)oxazine]; 5-methoxy-1,3,3-trimethylspiro[indoline-2,3′-[3H]naphtho[2,1-b]pyran]; 2,3,3-trimethyl-1-propyl-3H-indolium iodide and the like.

Photo-luminescent compounds can find use in a variety of color change mediums and formats. Photo-luminescent compounds can include, but are not limited to, a variety of materials. Greens, green blue and violet can be made with alkaline earth aluminates activated by rare earth ions. By way of example, strontium aluminate can be activated using europium (SrAl0₃:Eu). Visual wavelengths can include: green at 520 nm, blue-green at 505 nm, and blue at 490 nm. Red and orange colors can be generated with zinc sulfide.

Photochromic and photoluminescent dyes can be added in from 0.001% by weight to 80% by weight. More usually, the dye additive will find use in the range form 0.01% to 50% by weight. Usually, the additive will range in concentrations from 0.1% to 25%. Typically and most often, the colorant will be added in a range from 0.5% to 5%.

### Softening Compositions:

Softening components can be added to a composition to increase its pliability, plasticity, and flexibility. Softening agents can include, but are not limited to: mineral oil, petroleum jellies, natural and synthetic oils, vegetable oils, corn oil, canola oil, olive oil, various other extracted oils, powder compounds that provide slippage such as talc powder, long chain alcohols, glycerin oils, polyethylene glycols of various chain lengths, lanolin, soft bees wax and related natural waxes, gel compounds, and the like.

### Protective Oils and Coatings:

Protective coating materials can be added top the composition to provide a preserving effect on a surface to be cleansed and treated by the composition. Protective coatings can include, but are not limited to: ultraviolet light inhibitors such as nanometer particles, zinc oxide, para-aminobenzoate, soap oils such as Murphy's Soap Oil™ for wood surface care, carbarundum wax, Pledge™ compositions used for furniture care and related furniture waxes, oils used for lotions and related skin care products, and the like.

### Gelling and Texturizing Agents:

Several gelling and texturizing agents can be added to the composition to modify the texture and quality of the formulation. Examples include: glycerol mono-stearate, carboxypolymethylene as a gelling agent, methyl and propyl paraben, polysorbate, preferably polyoxyethylene, sorbitan monolaurate, fumed silica, thixotropic materials, polymer blends, acrylic powders, polyethylene powders, and the like.

### Petroleum Product and High Boiling Point Additives:

The addition of a particular petroleum based feed stock material can be used to sensitize the composition to the adsorption and pick of a like-kind dirt or grime. For example, the addition of a long chain greases can be utilized to facilitate the adsorption and entrapment of machine greases and oils. The composition can be tailored to a specific product or consumer application of interest.

The addition of high-boiling point organics can be added as sensitizers to facilitate the removal of difficult staining materials from surfaces. By way of example high staining substances like food dyes, coffee, tea, inks and the like can be more effectively removed by improving the bonding characteristics of the stain from the surface and facilitating the transport of the staining compound into the composition matrix.

The following examples are offered by way of illustration and not by way of limitation.

### Examples

### Example 1

### Polymer Blended Composition Formulation

An initial base composition was formulated by admixing 40% by weight poly (isobutylene-isoprene copolymer) as an active adhesive agent, 30% by weight powdered calcium carbonate as an inert filler, 22.5% by weight fine sifted talc, 2.5% silica powder (10-50 micron particle size) 2.5% by weight soy lecithin as an emulsifier, and 2.5% by weight glycerin oil. 100 kg initial base composition was heated to 250° F. and blended for 10 minutes and then poured into conveniently sized ingots and cooled for further use.

The blended formulation can be further processed into a final solventless cleaning composition by adding additional de-tackifying components, softening components and matrix lightening components. Ingots prepared above can be re-softened by heating to 250° F. 8% silicon oil (1000 centistokes), 1% by weight lightweight micro-spheres (Expancel™ DE 40), and 8% mineral oil (low liquid wax). The molten mixture was cooled and blended to 180° F. and molded in an aluminum mold into rod shapes. The composition rod shapes were allowed to cool to room temperature prior to use.

For usage, a 2 to 4 gram rod section was easily softened by hand through kneading and rolling it until the piece turned to a softened putty-like consistency. The soften consistency of the composition indicated that the composition had been adequately elevated in temperature for optimal cleaning activity. The rolling and kneading by hand immediately resulted in hand cleansing during the activation process. The composition could further be used for cleaning other dirty surfaces by using a back and forth rolling action directly on the contaminated area.

### Example 2

### Polymer Blended Composition Formulation with Thermochromic Indicator

An initial base composition was formulated by admixing 45% by weight poly (isobutylene-b-indene-b-isobutylene) as an active adhesive agent, 25% by weight powdered calcium carbonate as an inert filler, 25% by weight fine sifted talc, 2.5% by weight soy lecithin as an emulsifier, and 2.5% by weight glyceriri oil. 100 kg initial base composition was heated to 250° F. and blended for 10 minutes and then poured into conveniently sized ingots and cooled for further use.

The blended formulation can be further processed into a final solventless cleaning composition by adding additional de-tackifying components, softening components and matrix lightening components. Ingots prepared above can be re-softened by heating to 250° F. 8% silicon oil (1000 centistokes), 1% by weight lightweight micro-spheres (Expancel™ DE 40), 8% mineral oil (low liquid wax), and 2.0% aqueous thermochromic slurry (Matsui International AQ Ink™ type pink 27° C.) were thoroughly blended in to the molten mixture. The molten mixture was cooled and blended to 180° F. and molded in an aluminum mold into rod shapes. The composition rod shapes were allowed to cool to room temperature prior to use. Upon cooling to ambient room temperature, the composition turned to a pink/red coloration.

For usage, a 2 to 4 gram rod section was easily softened by hand through kneading and rolling it until the piece turned to a softened putty-like consistency. The color change indicated that the composition had been adequately elevated in temperature for optimal cleaning activity (at or above 27° C.). The rolling and kneading by hand immediately resulted in hand cleansing during the activation process. The composition could further be used for cleaning other dirty surfaces by using a back and forth rolling action directly on the contaminated area.

### Example 3

### Gum-Base Blended Composition Formulation

A pre-blended gum base formulation (L.A. Dryefus Co., Dreyco Base T LC, Dreyfus Base 37199, Dreyfus Base T 37199 or Dreyfus Base T 4098) was be further processed into a final solventless cleaning composition by adding additional de-tackifying components, softening components and matrix lightening components. Pellets of the above gum base formulation can be re-softened by heating to 250° F. 8% silicon oil (1000 centistokes), 3% by weight ethyvinyl acetate for increased elasticity (Atofina Inc. 33-400), 3% by weight lightweight micro-spheres (Expancel™ DE 40), and 8% mineral oil (low liquid wax), The molten mixture was cooled and blended to 180° F. and molded in an aluminum mold into rod shapes. The composition rod shapes were allowed to cool to room temperature prior to use.

For usage, a 2 to 4 gram rod section was easily softened by hand through kneading and rolling it until the piece turned to a softened putty-like consistency. The soften consistency of the composition indicated that the composition had been adequately elevated in temperature for optimal cleaning activity. The rolling and kneading by hand immediately resulted in hand cleansing during the activation process. The composition could further be used for cleaning other dirty surfaces by using a back and forth rolling action directly on the contaminated area.

### Example 4

### Gum-Base Blended Cleaning Composition Formulation with Thermochromic Indicator

A pre-blended gum base formulation (L.A. Dryefus Co., Dreyco Base T LC, Dreyfus Base 37199, Dreyfus Base T 37199 or Dreyfus Base T 4098) was be further processed into a final solventless cleaning composition by adding additional de-tackifying components, softening components and matrix lightening components. Pellets of the above gum base formulation can be re-softened by heating to 250° F. 8% silicon oil (1000 centistokes), 3% by weight ethyvinyl acetate for increased elasticity (Atofina Inc. 33-400), 3% by weight lightweight micro-spheres (Expancel™ DE 40), 8% mineral oil (low liquid wax), and 2.0% aqueous thermochromic slurry (Matsui International AQ Ink™ type fast blue 27° C.). The molten mixture was cooled and blended to 180° F. and molded in an aluminum mold into rod shapes. The composition rod shapes were allowed to cool to room temperature prior to use. The rods turned blue upon cooling to room temperature.

For usage, a 2 to 4 gram rod section was easily softened by hand through kneading and rolling it until the piece turned to a softened putty-like consistency. The soften consistency and color change of the composition indicated that the composition had been adequately elevated in temperature for optimal cleaning activity. The rolling and kneading by hand immediately resulted in hand cleansing during the activation process. The composition could further be used for cleaning other dirty surfaces by using a back and forth rolling action directly on the contaminated area.

### Example 5

### Gum-Base Blended Hand Cleaning/Softening Composition for Hands and Skin with Thermochromic Indicator

A pre-blended gum base formulation (L.A. Dryefus Co., Dreyco Base T LC, Dreyfus Base 37199, Dreyfus Base T 37199 or Dreyfus Base T 4098) was be further processed into a final solventless cleaning composition by adding additional de-tackifying components, softening components and matrix lightening components. Pellets of the above gum base formulation can be re-softened by heating to 250° F. 9% silicon oil (1000 centistokes), 3% by weight ethyvinyl acetate for increased elasticity (Atofina Inc. 33-400), 2% by weight lightweight micro-spheres (Expancel™ DE 40), 8% mineral oil (low liquid wax), 5% by weight hand lotion (Lubriderm™), and 2.0% aqueous thermochromic slurry (Matsui International AQ Ink™ type fast blue 27° C.). The molten mixture was cooled and blended to 180° F. and molded in an aluminum mold into rod shapes. The composition rod shapes were allowed to cool to room temperature prior to use. The rods turned blue upon cooling to room temperature.

For usage, a 2 to 4 gram rod section was easily softened by hand through kneading and rolling it until the piece turned to a softened putty-like consistency. The soften consistency and color change of the composition indicated that the composition had been adequately elevated in temperature for optimal cleaning activity. The rolling and kneading by hand immediately resulted in hand cleansing during the activation process. Addition of a hand lotion to the formulation provides an enhanced hand-softening characteristic provided by the lotion.

### Example 6

### Gum-Base Blended Hand/Skin Cleaning/Sunscreen Application Composition for Hands and Skin with Photochromic UV Indicator Dye

A pre-blended gum base formulation (L.A. Dryefus Co., Dreyco Base T LC, Dreyfus Base 37199, Dreyfus Base T 37199 or Dreyfus Base T 4098) was be further processed into a final solventless cleaning composition by adding additional de-tackifying components, softening components and matrix lightening components. Pellets of the above gum base formulation can be re-softened by heating to 250° F. 9% silicon oil (1000 centistokes), 3% by weight ethyvinyl acetate for increased elasticity (Atofina Inc. 33-400), 2% by weight lightweight micro-spheres (Expancel™ DE 40), 8% mineral oil (low liquid wax), 5% by weight sunscreen hand lotion (Walgreens™ Brand, SPF 30), and 2.0% aqueous photochromic slurry (Matsui International Photopia Ink™ type clear to red). The molten mixture was cooled and blended to 180° F. and molded in an aluminum mold into rod shapes. The composition rod shapes were allowed to cool to room temperature prior to use. The rods turned blue upon cooling to room temperature.

For usage, a 2 to 4 gram rod section was easily softened by hand through kneading and rolling it until the piece turned to a softened putty-like consistency. The soften consistency and color change of the composition indicated that the composition had been adequately elevated in temperature for optimal cleaning activity. The rolling and kneading by hand immediately resulted in hand cleansing during the activation process. Addition of a sunscreen component to the formulation provides a UV skin protection property.

### Example 7

### Gum-Base Blended Fabric and Carpet Cleaning Composition with Thermochromic Indicator

A pre-blended gum base formulation (L.A. Dryefus Co., Dreyco Base T LC, Dreyfus. Base 37199, Dreyfus Base T 37199 or Dreyfus Base T 4098) was be further processed into a final solventless cleaning composition by adding additional de-tackifying components, softening components and matrix lightening components. Pellets of the above gum base formulation can be re-softened by heating to 250° F. 9% silicon oil (1000 centistokes), 3% by weight ethyvinyl acetate for increased elasticity (Atofina Inc. 33-400), 8% mineral oil, 2% by weight lightweight micro-spheres (Expancel™ DE 40), 5% Spray & Wash™ (viscous fabric pre-cleaning product), and 2.0% aqueous thermochromic slurry (Matsui International AQ Ink™ type brilliant green 27° C.). The molten mixture was cooled and blended to 180° F. and molded in an aluminum mold into rod shapes. The composition rod shapes were allowed to cool to room temperature prior to use. The rods turned blue upon cooling to room temperature.

For usage, a 2 to 4 gram rod section was easily softened by hand through kneading and rolling it until the piece turned to a softened putty-like consistency. The soften consistency and color change of the composition indicated that the composition had been adequately elevated in temperature for optimal cleaning activity. The rolling and kneading by hand immediately resulted in fabric cleansing during the activation process. Loose dirt and grease types were removed using the composition.

### Example 8

### Hydrocarbon-Wax Based Cleaning Composition for Cleaning Furniture

An alternate basic composition comprises a malleable hydrocarbon wax composition that is soft and putty or clay-like when deformed by hand. The composition can be contain feed-stock petroleum distillates, Typical suppliers include Exxon Mobile, International Group Inc., Clams Specialty Products Inc., Accu-Blend Corporation, and other specialty manufacturers or formulators of wax-based products.

Composition formulation was accomplished by melting 98.5% by weight soft paraffin wax (IGI-2206 from Claris Specialty Products) at 200° F. in a convection oven. 0.5% by weight thermochromic powder (25° C. red thermochromic powder from Color Change Corporation) was added to the molten solution and mixed by sonicating the pigment for 1 minute. The solution was allowed to cool to 150° F., but maintained in its mixed molten state. 1.0% by weight fumed silica was mixed carefully into the molten solution. The final composition was cast into rod-like ingots in an aluminum mold and allowed to cool to room temperature.

For cleaning wood and other hard surfaces, a 2 to 4 gram rod section was easily softened by hand through kneading and rolling it until the piece turned to a softened putty-like consistency. The soften consistency and color change of the composition indicated that the composition had been adequately elevated in temperature for optimal cleaning activity. The rolling and kneading by hand over a wood or hard surface immediately resulted in cleaning and shining the surface. Loose dirt and grease types were removed using the composition.

### Example 9

### Hydrocarbon-Wax Based Cleaning Composition for Hand Cleaning, Anti-Bacterial Activity and Thermochromic Indicating Means

The basic formulation described above in Example 9. was further modified with a talc powder and an anti-bacterial ointment to create a therapeutic cleaning and anti-bacterial active composition.

Composition formulation was accomplished by melting 97.5% by weight soft paraffin wax (IGI-2206 from Claris Specialty Products) at 200° F. in a convection oven. 0.5% by weight thermochromic powder (25° C. red thermochromic powder from Color Change Corporation), 1% by weight talc powder, and 1% by weight anti-bacterial ointment (Neosporin Antibacterial Cream) was added to the molten solution and mixed by sonicating the pigment for 1 minute. The solution was allowed to cool to 150° F., but maintained in its mixed molten state. The final composition was cast into rod-like ingots in an aluminum mold and allowed to cool to room temperature.

For cleaning and providing antibacterial therapy to hands, a 2 to 4 gram rod section was easily softened by hand through kneading and rolling it until the piece turned to a softened putty-like consistency. The soften consistency and color change of the composition indicated that the composition had been adequately elevated in temperature for optimal cleaning activity. The rolling and kneading by immediately resulted in cleaning and shining the surface. Loose dirt and grease types were removed and a thin layer of antibacterial compounds were deposited by using the composition.

### Example 10

### Putty-Based Hand Cleanser for Bacterial Removal From Fingers and Skin Without Additional Sanitizing Agent

A pre-blended gum base formulation (L.A. Dryefus Co., Dreyco Base T LC, Dreyfus Base 37199, Dreyfus Base T 37199 or Dreyfus Base T 4098) was be was prepared according to Example 4 above. Pellets of the above gum base formulation can be re-softened by heating to 250° F. 9% silicon oil (1000 centistokes), 3% by weight ethyvinyl acetate for increased elasticity (Atofma Inc. 33-400), 3% by weight lightweight micro-spheres (Expancel™ DE 40), 9% mineral oil (low liquid wax), and 2.0% aqueous thermochromic slurry (Matsui International AQ Ink™ type fast blue 27° C.) were blended. The molten mixture was cooled and blended to 180° F. and molded in an aluminum mold into rod shapes. The rod shapes were allowed to cool to room temperature prior to use. The rods turned blue upon cooling to room temperature.

For usage, a 2 to 4 gram rod section was easily softened by hand through kneading and rolling it until the piece turned to a softened putty-like consistency. The soften consistency and color change of the composition indicated that the composition had been adequately elevated in temperature for optimal cleaning activity. The rolling and kneading by hand immediately resulted in hand cleansing during the activation process. Bacterial counts were assessed using prepared media plates and colony counting. Positive and negative control samples were taken from finger contact both before and after cleansing with the solventless hand sanitizer. Positive controls were accomplished using one minute cleaning cycles using the putty formulation. Continuous finger/skin contact was maintained throughout the cleansing cycle. Contact on sample and control plates was accomplished by direct contact of forger/skin to the auger surface. Prepared media plates (PML Microbiologics) that meet USP testing requirements were utilized for microbiological culturing and colony formation. Plate incubation was accomplished at 85° F. under sterile conditions. Plates were monitored for colony growth each 24 hours over a 14 day period. Finger contact samples prior to cleansing and sanitization with the cleansing putty showed a dramatic increase in colony counts compared to post cleansed fingers. Cleansed finger contact samples showed only back-ground colony counts compared to between 10 to 100 fold increase in colony counts for non-cleansed contact plates.

### Example 11

### Putty-Based Hand Cleanser for Bacterial Removal from Fingers and Skin with Added Germicide Agent

A pre-blended gum base formulation (L.A. Dryefus Co., Dreyco Base T LC, Dreyfus Base 37199, Dreyfus Base T 37199 or Dreyfus Base T 4098) was be was prepared according to Example 4 above. Pellets of the above gum base formulation can be re-softened by heating to 250° F. 9% silicon oil (1000 centistokes), 3% by weight ethyvinyl acetate for increased elasticity (Atofina Inc. 33-400), 3% by weight lightweight micro-spheres (Expancel™ DE 40), 9% mineral oil (low liquid wax), 0.2% benzalkonium chloride germicide (Alfa Aesar, a Johnson Matthey Company), and 2.0% aqueous thermochromic slurry (Matsui International AQ Ink™ type fast blue 27° C.) were blended. The molten mixture was cooled and blended to 180° F. and molded in an aluminum mold into rod shapes. The rod shapes were allowed to cool to room temperature prior to use. The rods turned blue upon cooling to room temperature.

For usage, a 2 to 4 gram rod section was easily softened by hand through kneading and rolling it until the piece turned to a softened putty-like consistency. The soften consistency and color change of the composition indicated that the composition had been adequately elevated in temperature for optimal cleaning activity. The rolling and kneading by hand immediately resulted in hand cleansing during the activation process. Bacterial counts were assessed using prepared media plates and colony counting. Positive and negative control samples were taken from finger contact both before and after cleansing with the solventless hand sanitizer. Positive controls were accomplished using one minute cleaning cycles using the putty formulation. Continuous finger/skin contact was maintained throughout the cleansing cycle. Contact on sample and control plates was accomplished by direct contact of finger/skin to the auger surface. Prepared media plates (PML Microbiologics) that meet USP testing requirements were utilized for microbiological culturing and colony formation. Plate incubation was accomplished at 85° F. under sterile conditions. Plates were monitored for colony growth each 24 hours over a 14 day period. Finger contact samples prior to cleansing and sanitization with the cleansing putty showed a dramatic increase in colony counts compared to post cleansed fingers. Cleansed finger contact samples using the included germicide showed only background colony counts compared to between 100 to 1000 fold increased colony counts for non-cleansed contact plates.

### Example 12

### Putty-Based Hand Cleanser for Bacterial Removal from Fingers and Skin witho Added Sanitizing Agent

A pre-blended gum base formulation (L.A. Dryefus Co., Dreyco Base T LC, Dreyfus Base 37199, Dreyfus Base T 37199 or Dreyfus Base T 4098) was be was prepared according to Example 4 above. Pellets of the above gum base formulation can be re-softened by heating to 250° F. 9% silicon oil (1000 centistokes), 3% by weight ethyvinyl acetate for increased elasticity (Atofina Inc. 33-400), 3% by weight lightweight micro-spheres (Expancel™ DE 40), 9% mineral oil (low liquid wax), and 2.0% aqueous thermochtomic slurry (Matsui International AQ Ink™ type fast blue 27° C.) were blended. The molten mixture was cooled and admixed with 5% ethanol as a sanitizing agent (Sigma Aldrich 100%), blended to a smooth consistency and molded in an aluminum mold into rod shapes. The rod shapes were allowed to cool to room temperature prior to use. The rods turned blue upon cooling to room temperature. The ethanol was retained and encapsulated in the putty matrix.

For usage, a 2 to 4 gram rod section was easily softened by hand through kneading and rolling it until the piece turned to a softened putty-like consistency. The soften consistency and color change of the composition indicated that the composition had been adequately elevated in temperature for optimal cleaning activity. The rolling and kneading by hand immediately resulted in hand cleansing during the activation process. Bacterial counts were assessed using prepared media plates and colony counting. Positive and negative control samples were taken from finger contact both before and after cleansing with the solventless hand sanitizer. Positive controls were accomplished using one minute cleaning cycles using the putty formulation. Continuous finger/skin contact was maintained throughout the cleansing cycle. Contact on sample and control plates was accomplished by direct contact of finger/skin to the auger surface. Prepared media plates (PML Microbiologics) that meet USP testing requirements were utilized for microbiological culturing and colony formation. Plate incubation was accomplished at 85° F. under sterile conditions. Plates were monitored for colony growth each 24 hours over a 14 day period. Finger contact samples prior to cleansing and sanitization with the cleansing putty showed a dramatic increase in colony counts compared to post cleansed fingers. Cleansed finger contact samples using the included sanitizer showed only background colony counts compared to between 100 to 1000 fold increased colony counts for non-cleansed contact plates. The composition has a distinct advantage over liquid/gel type hand sanitizers since the ethanol in the putty/cleanser matrix does not cause drying of skin and is not available for consumption by a user such as a child that might inadvertently consume an alcohol-based hand sanitizer.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A compliant liquid-free cleansing composition.

2. The compliant liquid-free cleaning composition according to claim 1, wherein said composition has a putty-like consistency.

3. The compliant liquid-free cleaning composition according to claim 1, wherein said composition is absorbant/adherant to contaminating species present on a surface.

4. The compliant liquid-free cleaning composition according to claim 1, wherein said composition comprises:
a viscous and adhesive base component;
an inert filler; and
an emulsifier.

5. The compliant liquid-free cleaning composition according to claim 4, wherein said base component is a rubber.

6. The compliant liquid-free cleaning composition according to claim 4, wherein said base component is a gum.

7. The compliant liquid-free cleaning composition according to claim 4, wherein said base component is a wax.

8. The compliant liquid-free cleaning composition according to claim 4, wherein said base component is a silicone.

9. The compliant liquid-free cleaning composition according to claim 1, wherein said composition comprises:
poly-isobutylene;
calcium carbonate; and
lecithin.

10. The compliant liquid-free cleaning composition according to claim 1, wherein said composition further comprises an abrasive.

11. The compliant liquid-free cleaning composition according to claim 1, wherein said composition further comprises a surfactant.

12. The compliant liquid-free cleaning composition according to claim 1, wherein said composition further comprises a germicidal agent.

13. The compliant liquid-free cleaning composition according to claim 1, wherein said composition further comprises a sanitizing agent.

14. The compliant liquid-free cleaning composition according to claim 1, wherein said composition further comprises a colorant.

15. The compliant liquid-free cleaning composition according to claim 1, wherein said composition further comprises a thermochromic colorant.

16. The compliant liquid-free cleaning composition according to claim 1, wherein said composition further comprises a fragrance.

17. A method of removing a contaminant from a surface, said method comprising contacting said surface with a composition according to claim 1 in a manner sufficient to remove said contaminant from said surface.

18. The method according to claim 17, wherein said method is a method of cleaning said surface.

19. The method according to claim 17, wherein said method further comprises delivering an agent to said surface.

20. The method according to claim 17, wherein said surface is a skin surface or a surface of an inanimate object.

21-29. (canceled)
